(19) United States
(12) Patent Application Publication
(10) Pub. No.: US 2006/0216277 A1
(43) Pub. Date: Sep. 28, 2006

(54) Insulin-producing bone marrow derived cells and methods of generating and using same

(75) Inventor: Shimon Efrat, Zikron-Yaakov (IL)

Correspondence Address: Martin D Moynihan;Prtsi Inc, PO Box 16446, Arlington, VA, 22215, US

(73) Assignee: RAMOT AT TEL AVIV UNIVERSITY LTD., TeL Aviv (IL)

(21) Appl. No.: 10/571,770

(22) PCT Filed: Sep. 2, 2004
(86) PCT No.: PCT/IL04/00790

§ 371 Date: Mar. 14, 2006

Related U.S. Application Data
(60) Provisional application No. 60/502,663, filed on Sep. 15, 2003.
(60) Provisional application No. 60/513,603, filed on Oct. 24, 2003.

(51) Int. Cl. A61K 48/00; C12N 5/08

(52) U.S. Cl. 424/93.21; 424/93.7; 435/372

(57) ABSTRACT

Insulin-producing bone marrow derived stem cells, and methods of generating and using same to reduce blood glucose levels in individuals.

### FIELD AND BACKGROUND OF THE INVENTION

The present invention relates to insulin-producing cells derived from bone marrow cells, and methods of generating and using same to reduce blood glucose levels in individuals.

Type 1 diabetes or juvenile-onset diabetes mellitus is an autoimmune disease which often strikes in childhood and results in a selective destruction of β-cells in the pancreas. As a result, type 1 diabetic patients suffer from loss of insulin, the polypeptide hormone responsible for the control of blood glucose level. Untreated diabetes can result in kidney failure, strokes, blindness and eventually death.

Current methods of treating type 1 diabetes involve periodic administration of insulin or its delivery using insulin pumps, such as that described for example in U.S. Pat Appl. No. 20030163223. However, in such cases blood glucose levels must be carefully monitored to ensure administration of appropriate amounts of insulin, since proper balancing of blood glucose levels requires insulin levels to be adjusted via a large number of physiological signals.

Thus, β-cell replacement has become the most promising approach for treating type 1 diabetes. However, since type 1 diabetes is an autoimmune condition, restoration of intracellular production of insulin should involve the replacement of β-cells with insulin-producing cells in a way that will avoid a recurring autoimmune response.

Indeed, transplantation of whole pancreas or isolated islets was shown to successfully replace the defective β-cells in type 1 diabetic patients [Shapiro A M et al., (2000). Islet transplantation in seven patients with type 1 diabetes mellitus using a glucocorticoid-free immunosuppressive regimen. New Engl. J. Med. 343: 230-238]. However, these approaches have been hampered due to limited donors of pancreatic tissue and since isolation of large quantities of pancreatic islets is both difficult and expensive.

To overcome these limitations, methods of expanding β-cells in culture have been developed.

Beta-cell cultures can be established by immortalizing mature post-mitotic β-cells. However, although this approach was successful in rodents, it faced difficulties with expansion of cells from human islets [Efrat, S. (2002). Trends in Molecular Medicine, 8: 334-339].

Alternatively, β-cells can be produced by differentiation of stem cells.

For example, spontaneous differentiation of both mouse and human embryonic stem cells (ESCs) resulted in a small percentage of insulin-producing cells [Assady S. et al., 2001. Insulin production by human embryonic stem cells. Diabetes 50: 1691-1697; Soria B. et al., 2000. Insulin-secreting cells derived from embryonic stem cells normalize glycemia in streptozotocin-induced diabetic mice. Diabetes 49: 157-162]. However, these cells produced low amounts of insulin, as compared with β-cells, and their potential use in transplantation has met with ethical objections, as well as concerns regarding the risk of teratoma formation.

Other stem cells which can be used for generating β-cells are fetal and adult tissue stem cells.

For example, the epithelium of the pancreatic duct serves as a source of cells capable of islet neogenesis in the adult, and may constitute the pancreatic stem cells, from which normal renewal of islets occurs throughout life. However, the use of these cells as a source for generation of insulin-producing cells is limited by their low expansion capacity in tissue culture and slow differentiation rate into insulin-producing cells.

Recent studies have shown that tissue stem cells are capable of reprogramming using dominant genes which activate a cascade of developmental events. Thus, mouse [Ferber S. et al. (2000). Pancreatic and duodenal homeobox gene 1 induces expression of insulin genes in liver and ameliorates streptozotocin-induced hyperglycemia Nat. Med. 6: 568-572] and Xenopus [Horb M E. Et al., (2003). Experimental conversion of liver to pancreas. Curr. Biol. 13: 105-115] liver cells, as well as rat enterocytes [Kojima H et al. (2002). Combined expression of pancreatic duodenal homeobox 1 and islet factor 1 induces immature enterocytes to produce insulin. Diabetes 51: 1398-1408] were shown to activate β-cell gene expression following the expression of pancreatic duodenal homeobox 1 (Pdx1), a homeobox factor which plays key roles in pancreas development and gene expression in mature β cells [Jonsson J. et al., (1994). Insulin-promoter-factor 1 is required for pancreas development in mice. Nature 371: 606-609].

In addition, cultured human fetal liver cells modified by the expression of the Pdx1 gene were shown to produce and store mature insulin in significant amounts, release it in response to physiological glucose levels and replace β-cell function in streptozotocin (STZ)-diabetic non-obese diabetic severe combined immunodeficiency (NOD-scid) mice [Zalzman M. et al., (2003). Reversal of hyperglycemia in mice by using human expandable insulin-producing cells differentiated from fetal liver progenitor cells. Proc Natl Acad Sci USA 100: 7253-7258]. These cells expressed multiple β-cell genes, as well as genes of other islet cells and the exocrine pancreas, and continued to express some hepatic genes. However, as in other cases of tissue transplantation, fetal liver cells are likely to induce allograft rejection, which can complicate the process of β-cell replacement.

To avoid allograft rejection, β-like cells can be generated from autologous stem cells, such as those present in the bone marrow (BM). BM stem cells are capable of self-renewal and as such can be repeatedly collected from autologous donors in order to accumulate large quantities of stem cells. In addition, BM stem cells can be expanded in culture under conditions retaining their self-renewal and pluripotential capacities [Ballas, C. B. et al. (2002). Adult bone marrow stem cells for cell and gene therapies: implications for greater use. J Cell Biochem Suppl. 38: 20-8].

BM stems cells are capable of differentiating into all hematopoietic cell lineages. As such, BM cell transplantation has become the desired approach for treating patients suffering from leukemia and other hematological nonmalignant disorders [see for example Khojasteh N H. Et al., (2002). Bone marrow transplantation for hematological disorders—Shiraz experience. Indian J Pediatr. 69: 31-2; Harden S V. et al., (2001). Total body irradiation using a modified standing technique: a single institution 7-year experience. Br J Radiol. 74: 1041-7; Saba N, Flaig T. (2002). Bone marrow transplantation for nonmalignant diseases. J Hematother Stem Cell Res. 11: 377-87].

Recent findings demonstrate that in addition to hematopoietic stem cells, the BM contains other stromal or mesenchymal stem cells capable of differentiating to various cell types [Jiang Y et al. (2002). Pluripotency of mesenchymal stem cells derived from adult marrow. Nature 418: 41-49].

As such, BM stem cells represent an almost unlimited source of cells suitable for cell replacement therapy. In addition, BM stem cells are capable of being targeted to the tissue of choice, especially in cases of injury [Chopp M, Li Y. (2002). Treatment of neural injury with marrow stromal cells. Lancet Neurol. 1: 92-100]. Moreover, new methods have been developed to avoid autoimmune responses following allogeneic BM transplants (BMI). Thus, BM cells containing a small number of T-cells were successfully injected into the BM cavity [i.e., intra-bone marrow (IBM)] of MRL/lpr mice, and treated mice survived for 2-years post-transplantation [Ikehara S. (2003). New strategies for allogeneic BMT Bone Marrow Transplant 32 Suppl 1: S73-5]. In addition, methods have been developed to reduce the accompanying toxicity following allogeneic BM transplantation [Beguin Y, Baron F. (2003). Minitransplants: allogeneic stem cell transplantation with reduced toxicity. Acta Clin Belg. 58: 37-45].

However, current attempts to utilize unmodified BM cells for O-cell replacement resulted in low frequency of donor insulin-positive cells in the pancreas [Hess D. et al., (2003). Bone marrow-derived stem cells initiate pancreatic regeneration. Nat Biotechnol. 21: 763-70]. In addition, exogenous regeneration of β-cells using unmodified BM cells would probably fail in an autoimmune environment such as that present in type 1 diabetes.

While reducing the present invention to practice, the present inventors have devised a novel methodology which can be utilized to generate insulin-producing cells from human BM stem cells. As is demonstrated herein, cell produced using such methodology are capable of reducing glucose blood level in vivo and can therefore be used for β-cell replacement and treatment of type 1 diabetes.

### SUMMARY OF THE INVENTION

According to one aspect of the present invention there is provided a method of generating insulin-producing cells comprising: (a) isolating and optionally culturing bone marrow stem cells to thereby obtain a bone marrow stem cell culture; and (b) expressing exogenous Pdx1 in cells of the bone marrow stem cell culture to thereby obtain insulin-producing cells.

According to another aspect of the present invention there is provided a method of generating insulin-producing cells comprising: (a) isolating and optionally culturing adult tissue stem cells to thereby obtain an adult tissue stem cell culture; and (b) expressing exogenous Pdx1 in cells of the adult tissue stem cell culture to thereby obtain insulin-producing cells.

According to yet another aspect of the present invention there is provided a method of reducing blood glucose levels in an individual comprising administering Pdx1-expressing bone marrow stem cells to the individual thereby reducing blood glucose levels in the individual.

According to further features in preferred embodiments of the invention described below, the Pdx1-expressing bone marrow stem cells are prepared by: (a) isolating and optionally culturing bone marrow stem cells to thereby obtain a bone marrow stem cell culture; and (b) expressing exogenous Pdx1 in cells of the bone marrow stem cell culture to thereby obtain Pdx1-expressing bone marrow stem cells.

According to still another aspect of the present invention there is provided a method of reducing blood glucose levels in an individual comprising: (a) isolating and optionally culturing bone marrow stem cells to thereby obtain a bone marrow stem cell culture; (b) expressing exogenous Pdx1 in cells of the bone marrow stem cell culture to thereby obtain Pdx1-expressing bone marrow stem cells; and (c) administering the Pdx1-expressing bone marrow stem cells to the individual thereby reducing blood glucose levels in the individual.

According to an additional aspect of the present invention there is provided a method of treating an individual having a disorder requiring 13-cell replacement, comprising administering Pdx1-expressing bone marrow stem cells to the individual thereby treating the individual.

According to flier features in preferred embodiments of the invention described below, the Pdx1-expressing bone marrow stem cells are prepared by: (a) isolating and optionally culturing bone marrow stem cells to thereby obtain a bone marrow stem cell culture; and (b) expressing exogenous Pdx1 in cells of the bone marrow stem cell culture to thereby obtain Pdx1-expressing bone marrow stem cells.

According to yet an additional aspect of the present invention there is provided a method of treating an individual having a disorder requiring O-cell replacement comprising: (a) isolating and optionally culturing bone marrow stern cells to thereby obtain a bone marrow stem cell culture; (b) expressing exogenous Pdx1 in cells of the bone marrow stem cell culture to thereby obtain Pdx1-expressing bone marrow stem cells; and (c) administering the Pdx1-expressing bone marrow stem cells to the individual thereby treating the individual.

According to still an additional aspect of the present invention there is provided a method of reducing blood glucose levels in an individual comprising administering Pdx1-expressing adult tissue stem cells to the individual thereby reducing blood glucose levels in the individual.

According to further features in preferred embodiments of the invention described below, the Pdx1-expressing adult tissue stem cells are prepared by: (a) isolating and optionally culturing adult tissue stem cells to thereby obtain an adult tissue stem cell culture; and (b) expressing exogenous Pdx1 in cells of the adult tissue stem cell culture to thereby obtain Pdx1-expressing adult tissue stem cells.

According to a further aspect of the present invention there is provided a method of reducing blood glucose levels in an individual comprising: (a) isolating and optionally culturing adult tissue stem cells to thereby obtain an adult tissue stem cell culture; (b) expressing exogenous Pdx1 in cells of the adult tissue stem cell culture to thereby obtain Pdx1-expressing adult tissue stem cells; and (c) administering the Pdx1-expressing adult tissue stem cells to the individual thereby reducing blood glucose levels in the individual.

According to yet a further aspect of the present invention there is provided a method of treating an individual having a disorder requiring 1-cell replacement, comprising administering Pdx1-expressing adult tissue stem cells to the individual thereby treating the individual.

According to further features in preferred embodiments of the invention described below, the Pdx1-expressing adult tissue stem cells are prepared by: (a) isolating and optionally culturing adult tissue stem cells to thereby obtain an adult tissue stem cell culture; and (b) expressing exogenous Pdx1 in cells of the adult tissue stem cell culture to thereby obtain Pdx1-expressing adult tissue stem cells.

According to still a further aspect of the present invention there is provided a method of treating an individual having a disorder requiring β-cell replacement comprising: (a) isolating and optionally culturing adult tissue stem cells to thereby obtain an adult tissue stem cell culture; (b) expressing exogenous Pdx1 in cells of the adult tissue stem cell culture to thereby obtain Pdx1-expressing adult tissue cells; and (c) administering the Pdx1-expressing adult tissue stem cells to the individual thereby treating the individual.

According to further features in preferred embodiments of the invention described below, the method further comprising a step of immortalizing the cells of the bone marrow stem cell culture.

According to still further features in the described preferred embodiments immortalizing is effected by expressing in the cells of the bone marrow stem cell culture a telomerase.

According to still further features in the described preferred embodiments culturing is effected under culturing conditions selected suitable for expansion of mesenchymal stem cells of the bone marrow stem cells.

According to still further features in the described preferred embodiments expressing in the cells the exogenous Pdx1 is effected by transfecting the cells with an expression vector including a polynucleotide encoding the Pdx1 positioned under the transcriptional control of a mammalian promoter.

According to still further features in the described preferred embodiments, the method further comprising a step of immortalizing the cells of the adult tissue stem cell culture.

According to still further features in the described preferred embodiments, immortalizing is effected by expressing in the cells of the adult tissue stem cell culture a telomerase.

According to still further features in the described preferred embodiments expressing in the cells the telomerase is effected by transfecting the cells with an expression vector including a polynucleotide encoding the telomerase positioned under the transcriptional control of a mammalian promoter.

According to still further features in the described preferred embodiments the adult tissue stem cells are derived from an adult tissue selected from the group consisting of adipose tissue, skin, kidney, liver, prostate, pancreas, intestine, and bone marrow.

According to still a further aspect of the present invention there is provided a cell culture comprising bone marrow stem cells genetically modified to express Pdx1.

According to still a further aspect of the present invention there is provided a cell culture comprising adult tissue stem cells genetically modified to express Pdx1.

According to still further features in the described preferred embodiments the cells are capable of producing insulin.

According to still further features in the described preferred embodiments the cells are capable of expressing pancreatic beta cell genes.

According to still further features in the described preferred embodiments the bone marrow stem cells are immortalized.

According to still further features in the described preferred embodiments the bone marrow stem cells are genetically modified to express a telomerase.

According to still further features in the described preferred embodiments the adult tissue stem cells are immortalized.

According to still further features in the described preferred embodiments the adult tissue stem cells are genetically modified to express a telomerase.

The present invention successfully addresses the shortcomings of the presently known configurations by providing insulin-producing bone marrow stern cells, and methods of generating and using same to reduce blood glucose levels in individuals.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c schematically illustrate various approaches for β-cell replacement using bone marrow (BM) derived stem cells. FIG. 1a illustrates putative natural homing of autologous BM stem cells to the pancreas, representing a possible endogenous regeneration pathway, which likely fails in type 1 diabetes due to recurring autoimmunity. FIG. 1b illustrates transplantation of in vitro differentiated β-like cells from autologous BM cells in an extra-pancreatic site such as the liver. FIG. 1c illustrates transplantation of β-like cells differentiated in vitro from allogeneic BM cells in the liver, along with the transplantation of undifferentiated BM cells from the same donor to reconstitute BM in an irradiated recipient.

FIGS. 2a-b illustrate the DNA constructs used for transfecting the bone marrow stem cells. FIG. 2a is a schematic illustration of the hTERT/GFP DNA construct including the cytomegalovirus promoter and enhancer (CMVₚ), the catalytic subunit of human telomerase cDNA (hTERT, SEQ D NO:1), an internal ribosomal entry site (IRES) and the cDNA encoding enhanced green fluorescence protein (GFP). FIG. 2b is a schematic illustration of the Pdx1 cDNA construct including the phosphoglycerate kinase promoter (PGKₚ), the rat pancreatic duodenal homeobox 1 cDNA (Pdx1, SEQ ID NO:2), an IRES sequence, the neomycin resistance gene (Neo) and the woodchuck hepatitis virus posttranscriptional modification element (WHV).

FIGS. 3a-c illustrate the expression of recombinant GFP in the hTERT/GFP transfected BM cells. FIG. 3a is a FACS histogram depicting specific expression of the recombinant GFP protein in transfected BM cells; FIG. 3b is a FACS histogram depicting the selection of GFP-positive cells using FACS-gating analysis; FIG. 3c depicts immunofluorescence micrographs illustrating the selection of GFP-positive cells following FACS-gating. Shown are fluorescent images of transfected BM cells viewed under DAPI, GFP and Rhodamine filters before (Pre-FACS) or following (Post-FACS) the selection of GFP-positive cells using FACS-gating.

FIG. 4 illustrates the expression of recombinant hTERT in transfected BM cells. RT-PCR reactions were performed on RNA samples from untransduced BM cells (lane 3) or from BM cells transfected with the hTERT/GFP plasmid (lane 4). The specificity of the reaction was determined in the absence of RNA (lame 2) or in the presence of an hTERT/GFP plasmid DNA (lane 1).

FIG. 5 is an immunofluorescence micrograph illustrating the expression of insulin protein in Pdx1-expressing BM cells. Shown is a fluorescent image of a human BM cell transfected with both the hTERT/GFP and Pdx1 plasmids and immunostained using an antibody directed against insulin.

FIG. 6 is an RT-PCR determination of gene expression in human islets and transfected BM cells. Shown is one representative experiment out of three reproducible experiments using BM cells from three different donors. RT-PCR reactions were performed on RNA samples extracted from human pancreatic islets and transfected BM cells. Lane 1—human islets; lane 2—RT-PCR reaction devoid of RNA; lane 3—human BM cells transfected with the hTERT/GFP plasmid alone; lane 4—human BM cells transfected with both the hTERT/GFP and Pdx1 plasmids.

FIG. 7 illustrates the effect of transplantation of human BM cells transfected with both the hTERT/GFP and Pdx1 plasmids on blood glucose levels in vivo. Cells were injected into the tail vein of a STZ-diabetic immunodeficient (NOD-scid) mouse. Shown are blood glucose levels measured following cell transplantation.

FIG. 8 is a schematic illustration of the tet-HNF6/Neo DNA construct. The hepatocyte nuclear factor 6 (BNF6) cDNA (SEQ ID NO:51) was placed under control of a minimal promoter (CMV promoter) and the tet-operator sequences (tat-op) and upstream of an SV40 polyadenylation element (SV40 Aₙ) and a neomycin resistance gene (Neo) of the pUHD10-3 vector (a gift of H. Bujard, see Efrat S, et al., 1995, Proc. Natl. Acad. Sci. USA 92: 3576-3580).

FIG. 9 is a schematic illustration of the tet-NeuroD/Hyg construct. The neurogenic differentiation 1 (NeuroD) cDNA (SEQ ID NO:52) was placed under control of a minimal promoter (CMV promoter) and the tet-operator sequences (tet-op) and upstream of an SV40 polyadenylation element (Aₙ) of the PTRE2hyg vector (Clontech) which also includes the hygroycin resistance gene (Hyg) under the control of the SV40 promoter (SV40ₚ) and the ColiE1 origin of replication (pUC Ori).

FIG. 10 is a schematic illustration of the tet-Ngn3/Neo construct. The neurogenin 3 (Ngn3) cDNA (SEQ ID NO:53) was placed under control of a minimal promoter (CMV promoter) and the tet-operator sequences (tet-op) and upstream of an IRES (internal ribosome entry site) element of the pIRES (Clontech) vector, which also includes a neomycin resistance gene (Neo) and a woodchuck hepatitis virus posttranscriptional modification element (WHV).

FIG. 11 is a schematic illustration of the tet-Pdx1/Hyg construct. The Pdx1 cDNA (SEQ ID NO:2) was placed under control of a minimal promoter and the tet-operator sequences (tet-op) and upstream of a β-globin polyadenylation element (β-globin Aₙ) of the PTRE2Hyg vector (Clontech), which also includes the hygroycin resistance gene (Hyg).

FIG. 12 is a schematic illustration of the CMV-rtTA/Bla construct [PcDNA6/TR (Invitrogen)] containing the blasticidin resistance gene under the control of the SV40 promoter (SV40ₚ) and the reverse tetracycline transactivator (rtTA) under the control of the CMB promoter (CMVₚ).

FIG. 13 is a schematic illustration of the CMV-tTA/Puro construct. The puromycin resistance gene (Puro) under the control of the SV40 promoter (SV40ₚ) was introduced into the pUHD15-1 vector [a gift of H. Bujard; see Efrat S, et al., 1995, (Supra)] containing the tet-off tetracycline transactivator (tTA) under control of the CMV promoter (CMVₚ).

FIG. 14 is a schematic illustration of the CMV-hTERT/Zeo construct. The human telomerase reverse transcriptase (hTERT) cDNA (SEQ ID NO:1) was placed under control of the CMV promoter and upstream of a bovine growth hormone polyadenylation signal (BGH Aₙ) of the PcDNA3.1/Zeo vector (Invitrogen), which also includes the Zeomycin resistance gene (Zeo) under the control of the SV40 promoter (SV40ₚ) and polyadenylation element (SV40 Aₙ).

FIG. 15 is an RT-PCR analysis depicting the expression of Pdx1 under the tet-off transactivator, expression system. RNA was extracted from BM cells transiently co-transfected with the CMV-tTA/Puro and tet-Pdx1/Hyg plasmids which were incubated in the presence or absence of 0.5 μg/ml Doxycycline (Dox+/−) and was subjected to RT-PCR analysis using the rat Pdx1 forward (SEQ ID NO:3) and reverse (SEQ ID NO:4) PCR primers. BTP=positive control of bone marrow cells transfected with a constitutively expressed Pdx1 vector, Mix=negative control of the RT-PCR reaction mixture (without RNA). Note the high expression level of Pdx1 RNA in cells transfected with the tet-off transactivator expression system in the absence of Doxycycline.

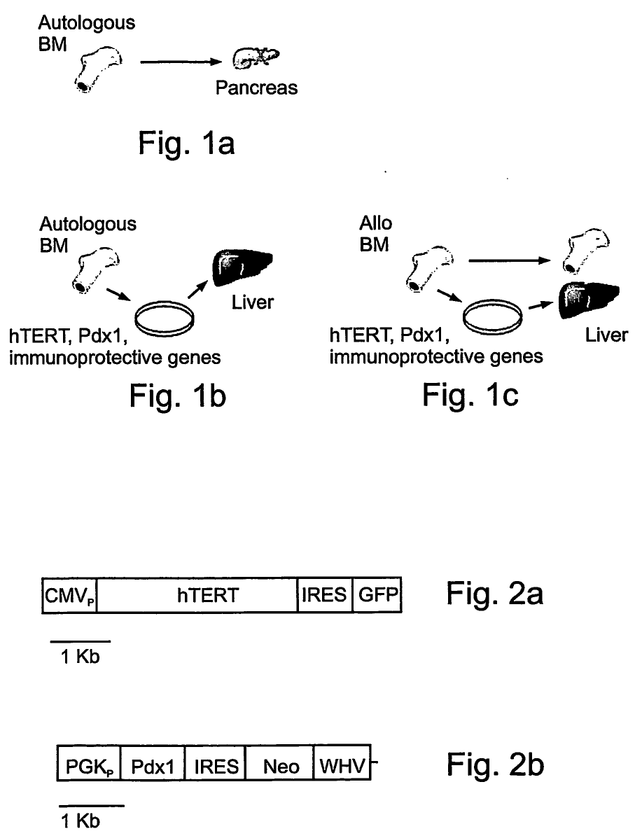

### DESCRIPTION OF THE PREFERRED EMBODIMENTS

The present invention is of insulin-producing BM cells and of methods of generating and using same in reducing blood glucose levels. Specifically, the methods and cells of the present invention can be used in cell replacement therapy of, for example, type 1 diabetes.

The principles and operation of the methods of reducing glucose blood levels according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Type 1 diabetes is an autoimmune disease characterized by selective destruction of insulin-producing cells (β-cells) in the pancreas. Thus, type 1 diabetes patients are dependent on periodic administration of exogenous insulin However, it is well known that for proper regulation of blood glucose levels, and to avoid complications resulting from uncontrolled diabetes, such as kidney failure, strokes, blindness and eventual death, insulin should be produced by cells capable of adjusting the amounts of secreted hormone in response to multiple physiological signals. Thus, at present, P cell replacement therapy is considered the most promising approach for treating type 1 diabetes.

Although pancreas or islet transplantation was shown to successfully replace defective β-cells in type 1 diabetic patients [Shapiro A M et al., (2000). New Engl. J. Med. 343: 230-238], these methods are limited by shortage of donor tissues.

To overcome these limitations, β-cells have been differentiated in tissue culture from embryonic stem cells (ESCs) or fetal liver stem cells. However, studies have shown that β-cells differentiated from ESCs produce low amounts of insulin [Assady S. et al., 2001. Diabetes 50: 1691-1697; Soria B. et al., 2000. Diabetes 49: 157-162] while their potential use in transplantation has met with ethical objections, as well as concerns regarding the risk of teratoma formation. In addition, differentiated β-cells from both human fetal liver cells [Zalzman M. et al., (2003). Proc Natl Acad Sci USA 100: 7253-7258] and human ESCs are likely to induce allograft rejection when transplanted in patients.

While reducing the present invention to practice, the present inventors have devised a novel approach for generating insulin-producing cells from autologous stem cells. As is shown in Example 2 of the Examples section which follows, transplantation of the insulin-producing cells of the present invention corrected insulin deficiency in a streptozotocin (STZ)-diabetic immunodeficient (NOD-scid) mouse.

Thus, according to the present invention there is provided a method of generating insulin-producing cells.

The method is effected by first isolating and optionally culturing stem cells to thereby obtain a stem cell culture and then expressing exogenous Pdx1 in the cells of the stem cell culture to thereby obtain the insulin-producing cells of the present invention.

As used herein, the phrase “insulin-producing cells” refers to cells expressing insulin polypeptides or peptides derived therefrom. Normally, insulin is produced and secreted by β-cells in the pancreas in response to physiological signals. Insulin-producing cells of the present invention are therefore β-like cells which produce insulin and secrete it preferably in response to physiological signals.

As used herein, the phrase “stem cells” refers to cells which are capable of remaining in an undifferentiated state (i.e. “pluripotent stem cells”) for extended periods of time in culture until induced to differentiate into other cell types having a particular, specialized function (i.e., “fully differentiated” cells).

The stem cells of the present invention can be adult tissue stem cells. As used herein, “adult tissue stem cells” refers to any stem cell derived from the postnatal animal (especially the human). The adult stem cell is generally thought to be a multipotent stem cell, capable of differentiation into multiple cell types. Adult stem cells can be derived from an adult tissue such as adipose tissue, skin, kidney, liver, prostate, pancreas, intestine, and bone marrow.

Methods of isolating adult tissue stem cells are known in the arts and include, for example, those disclosed by Alison, M. R. [Tissue-based stem cells: ABC transporter proteins take center stage. J Pathol. 2003 200(5): 547-50], Cal, J. et al., [Identifying and tracking neural stem cells. Blood Cells Mol Dis. 2003 31(1): 18-27] and Collins, A. T. et al., [Identification and isolation of human prostate epithelial stem cells based on alpha(2)beta(1)-integrin expression. J Cell Sci. 2001; 114(Pt 21): 3865-72].

Generally, isolation of adult tissue stem cells is based on the discrete location (or niche) of each cell type included in the adult tissue, i.e., the stem cells, the transit amplifying cells and the terminally differentiated cells [Potten, C. S. and Morris, R. J. (1988). Epithelial stem cells in vivo. J. Cell Sci. Suppl. 10, 45-62]. Thus, an adult tissue such as, for example, prostate tissue is digested with Collagenase and subjected to repeated unit gravity centrifugation to separate the epithelial structures of the prostate (e.g., organoids, acini and ducts) from the stromal cells. Organoids are then disaggregated into single cell suspensions by incubation with Trypsin/EDTA (Life Technologies, Paisley, UK) and the basal, CD44-positive, stem cells are isolated from the luminal, CD57-positive, terminally differentiated secretory cells, using anti-human CD44 antibody (clone G4426; Pharmingen, Becton Dickinson, Oxford, UK) labeling and incubation with MACS (Miltenyi Biotec Ltd, Surrey, UK) goat anti-mouse IgG microbeads. The cell suspension is then applied to a MACS column and the basal cells are eluted and re-suspended in WAJC 404 complete medium [Robinson, E. J. et al. (1998). Basal cells are progenitors of luminal cells in primary cultures of differentiating human prostatic epithelium Prostate 37, 149-160].

Since basal stem cells can adhere to basement membrane proteins more rapidly than other basal cells [Jones, P. H. et al. (1995). Stem cell patterning and fate in human epidermis. Cell 60, 83-93; Shinohara, T., et al. (1999). β1- and α6-integrin are surface markers on mouse spermatogonial stem cells. Proc. Natl. Acad. Sci. USA 96, 5504-5509] the CD44 positive basal cells are plated onto tissue culture dishes coated with either type I collagen (52 μg/ml), type IV collagen (88 μg/ml) or laminin 1 (100 μg/ml; Biocoat®, Becton Dickinson) previously blocked with 0.3% bovine serum albumin (fraction V, Sigma-Aldrich, Poole, UK) in Dulbecco's phosphate buffered saline (PBS; Oxoid Ltd, Basingstoke, UK). Following 5 minutes, the tissue culture dishes are washed with PBS and adherent cells, containing the prostate tissue basal stem cells are harvested with trypsin-EDTA.

Preferably, the stem cells utilized by the present invention are BM-derived stem cells including hematopoietic, stromal or mesenchymal stem cells (Dominici, M et al., 2001. Bone marrow mesenchymal cells: biological properties and clinical applications. J. Biol. Regul. Homeost. Agents. 15: 28-37). BM-derived stem cells may be obtained from iliac crest, femora, tibiae, spine, rib or other medullar spaces.

Of the above described BM-derived stem cells, mesenchymal stem cells are the formative pluripotent blast cells, and as such are preferred for use with the present invention. Mesenchymal stem cells give rise to one or more mesenchymal tissues (e.g., adipose, osseous, cartilaginous, elastic and fibrous connective tissues, myoblasts) as well as to tissues other than those originating in the embryonic mesoderm (e.g., neural cells) depending upon various influences from bioactive factors such as cytokines. Although such cells can be isolated from embryonic yolk sac, placenta, umbilical cord, fetal and adolescent skin, blood and other tissues, their abundance in the BM far exceeds their abundance in other tissues and as such isolation from BM is presently preferred.

As is mentioned hereinabove, and according to preferred embodiments of the present invention, the stem cells are cultured prior to expressing the exogenous Pdx1 gene therein.

When BM-derived stem cells are utilized, such cells are preferably cultured under conditions selected suitable for expansion of mesenchymal stem cells.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

Preferably, mesenchymal stem cell cultures are generated by diluting BM aspirates (usually 20 ml) with equal volumes of Hank's balanced salt solution (HBSS; GIBCO Laboratories, Grand Island, N.Y., USA) and layering the diluted cells over about 10 ml of a Ficoll column (Ficoll-Paque; Pharmacia, Piscataway, N.J., USA). Following 30 minutes of centrifugation at 2,500×g, the mononuclear cell layer is removed from the interface and suspended ill HBSS. Cells are then centrifuged at 1,500×g for 15 minutes and resuspended in a complete medium (MEM, a medium without deoxyribonucleotides or ribonucleotides; GIBCO); 20% fetal calf serum (FCS) derived from a lot selected for rapid growth of MSCs (Atlanta Biologicals, Norcross, Ga.); 100 units/ml penicillin (GIBCO), 100 μg/ml streptomycin (GIBCO); and 2 mM L-glutamine (GIBCO). Resuspended cells are plated in about 25 ml of medium in a 10 cm culture dish (Corning Glass Works, Corning, N.Y.) and incubated at 37° C. with 5% humidified CO₂. Following 24 hours in culture, nonadherent cells are discarded, and the adherent cells are thoroughly washed twice with phosphate buffered saline (PBS). The medium is replaced with a fresh complete medium every 3 or 4 days for about 14 days. Adherent cells are then harvested with 0.25% trypsin and 1 mM EDTA (Trypsin/EDTA, GIBCO) for 5 min at 37° C., replated in a 6-cm plate and cultured for another 14 days. Cells are then trypsinized and counted using a cell counting device such as for example, a hemocytometer (Hausser Scientific, Horsham, Pa.). Cultured cells are recovered by centrifugation and resuspended with 5% DMSO and 30% FCS at a concentration of 1 to 2×10⁶ cells per ml. Aliquots of about 1 ml each are slowly frozen and stored in liquid nitrogen.

To expand the mesenchymal stem cell fraction, frozen cells are thawed at 37° C., diluted with a complete medium and recovered by centrifugation to remove the DMSO. Cells are resuspended in a complete medium and plated at a concentration of about 5,000 cells/cm². Following 24 hours in culture, nonadherent cells are removed and the adherent cells are harvested using Trypsin/EDTA, dissociated by passage through a narrowed Pasteur pipette, and preferably replated at a density of about 1.5 to about 3.0 cells/cm². Under these conditions, MSC cultures can grow for about 50 population doublings and be expanded for about 2000 fold [Colter D C., et al. Rapid expansion of recycling stem cells in cultures of plastic-adherent cells from human bone marrow. Proc Nail Acad Sci USA. 97: 3213-3218, 2000].

MSC cultures utilized by the present invention preferably include three groups of cells which are defined by their morphological features: small and agranular cells (referred to as RS-1, hereinbelow), small and granular cells (referred to as RS-2, hereinbelow) and large and moderately granular cells (referred to as mature MSCs, hereinbelow). The presence and concentration of such cells in culture can be assayed by identifying a presence or absence of various cell surface markers, by using, for example, immunofluorescence, in situ hybridization, and activity assays.

When MSCs are cultured under the culturing conditions of the present invention they exhibit negative staining for the hematopoietic stem cell markers CD34, CD11B, CD43 and CD45. A small fraction of cells (less than 10%) are dimly positive for CD31 and/or CD38 markers. In addition, mature MSCs are dimly positive for the hematopoietic stem cell marker, CD117 (c-Kit), moderately positive for the osteogenic MSCs marker, Stro-1 [Simmons, P. J. & Torok-Storb, B. (1991). Blood 78, 5562] and positive for the thymocytes and peripheral T lymphocytes marker, CD90 (Thy-1). On the other hand, the RS-1 cells are negative for the CD117 and Stro1 markers and are dimly positive for the CD90 marker, and the RS-2 cells are negative for all of these markers.

As is mentioned hereinabove, following isolation and optional culturing, the stem cells of the present invention are transfected with an expression vector which is designed for expressing exogenous pancreatic duodenal homeobox 1 (Pdx1) in such cells.

To express Pdx1 in mammalian cells, a polynucleotide encoding Pdx1 is ligated into an expression vector under the control of a promoter suitable for mammalian cell expression.

As used herein “a polynucleotide encoding the Pdx1” refers to genomic or complementary polynucleotide sequence which encodes the pancreatic duodenal homeobox 1 protein. Examples of Pdx1 include rat Pdx1 (GenBank Accession Nos: NP_{—}074043, P52947), homo sapiens Pdx1 (GenBank Accession Nos: NP_{—}000200, P52945, AAB88463), zebrafish Pdx1 (GenBank Accession No: NP_{—}571518), mouse Pdx1 (GenBank Accession Nos: NP_{—}032840, P52946) and golden hamster Pdx1 (GenBank Accession Nos: P70118, AAB18252).

Coding sequences information for Pdx1 is available from several databases including the GenBank database available through http://www4.ncbi.nlm.nih.gov/. Examples of coding sequences which can be ligated into the expression vector described above, include, but are not limited to, human (GenBank Accession No: NM_{—}000209), rat (GenBank Accession No: NM_{—}022852), mouse (GenBank Accession No: NM_{—}008814) and zebrafish (GenBank Accession No: NM_{—}131443) cDNA sequences.

It will be appreciated that the nucleic acid expression construct of the present invention can also utilize Pdx1 homologues which exhibit the desired activity. Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to SEQ ID NO:2, as determined using the BestFit software of the Wisconsin sequence analysis package, utilizing the Smith and Waterman algorithm, where gap weight equals 50, length weight equals 3, average match equals 10 and average mismatch equals −9.

As is mentioned hereinabove, to enable mammalian cell expression, the expression vector of the present invention includes a promoter sequence for directing transcription of the polynucleotide sequence in a mammalian cell in a constitutive or inducible manner.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the hypoxia-inducible factor 1 (HIF-1) promoter (Rapisarda, A, et al., 2002. Cancer Res. 62: 4316-24) and the tetracycline-inducible promoter (Srour, M. A., et al., 2003. Thromb. Haemost. 90: 398-405).

The expression vector of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HWV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of Pdx1 mRNA translation Two distinct sequence elements are required for accurate and efficient polyadenylation: GU or U rich sequences located downstream from the polyadenylation site and a highly conserved sequence of six nucleotides, AAUAAA, located 11-30 nucleotides upstream, Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the vial genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A⁺, pMTO10/A⁺, pMAMneo-5, baculovirus pDSVE, and any other vector allowing expression of proteins under the direction of the SVAO early promoter, SV40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein For example, bone marrow cells can be targeted using the human T cell leukemia virus type I (HTLV-I).

Recombinant viral vectors are useful for in vivo expression of Pdx-1 since they offer advantages such as lateral infection and targeting specificity. Lateral infection is inherent in the life cycle of, for example, retrovirus and is the process by which a single infected cell produces many progeny varions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Since normal human cells, including adult tissue stem cells, are difficult to propagate for long periods in culture, the stem cells of the present invention are preferably immortalized to increase their replication capacity and viability in culture.

Thus, according to the present invention, the method described hereinabove further includes a step of immortalizing the cells of the stem cell culture.

As used herein, the phrase “immortalizing the cells” refers to providing the cells with immortalizing gene(s) which increase the life span of the cell, especially in culture under replicative growth conditions, such that the resulting cell line is capable of being passaged more times than the original primary cells.

According to preferred embodiments of the present invention, immortalizing is effected by expressing a telomerase in the cells of the stem cell culture, since it has been previously shown that mammalian cells undergo telomere shortening during replication [Harley C B, Futcher A B, Greider C W. Telomeres shorten during ageing of human fibroblasts. Nature 1990; 345:458-460].

Telomerase is a ribonucleoprotein enzyme that synthesizes one strand of the telomeric DNA using as a template a sequence contained within the RNA component of the enzyme. The ends of chromosomes have specialized sequences, termed telomeres, comprising tandem repeats of simple DNA sequences which in humans is 5′-TTAGGG. Apart from protecting ends of chromosomes telomeres have several other functions, the most important of which appear to be associated with replication, regulating the cell cycle clock and ageing. Progressive rounds of cell division shorten telomeres by 50-200 nucleotides per round.

The telomerase utilized by the present invention can be of any source including for example, homo sapiens (GenBank Accession No: NM_{—}003219), mouse (GenBaik Accession Nos: AF051911, AF073311), Xenopus laevis (GenBank Accession No: AF212299), golden hamster (GenBank Accession No: AF149012), Oryza Sativa (GenBank Accession No: AF288216) and arabidopsis thaliana (GenBank Accession No: AF135454).

Telomerase is expressed in the Pdx1 expressing stem cells of the present invention by introducing into these cells a second expression vector which includes a polynucleotide sequence encoding telomerase positioned under the transcriptional control of a mammalian promoter. Suitable expression vectors which can be utilized to express the telomerase gene are described hereinabove and in example 1 of the Examples section which follows.

It will be appreciated that telomerase expression can also be effected from the expression construct utilized for Pdx1 expression by utilizing an IRES sequence (Wu Q. et al., 2003. Virus Res. 93: 211-9) or by placing the telomerase coding sequence under the transcriptional control of a second promoter. Example 1 of the examples section which follows illustrates the use of an IRES sequence for co-expression of two sequences from a single vector.

As is further shown in Example 1 of the Examples section which follows, BM cells cultured according to the teachings of the present invention and transfected with the Pdx1 and hTERT DNA constructs are capable of producing high amounts of insulin.

Thus, the present invention provides a novel approach for generating insulin producing cells which are both effective in producing high amounts of insulin and also capable of being derived from autologous sources, such as the BM of the individual in need of insulin-producing cells.

Due to their insulin production capacity and availability, cells produced according to the teachings of the present invention can be utilized in cell replacement therapy of various diseases. Indeed, as is shown in Example 2 of the Examples section which follows, the Pdx1-expressing cells of the present invention effectively reduced blood glucose levels of STZ-diabetic immunodeficient (NOD-scid) mice when administered thereto.

Thus, according to another aspect of the present invention, there is provided a method of reducing blood glucose levels in an individual. The method is effected by administering the Pdx1-expressing cells of the present invention to the individual thereby reducing blood glucose levels in the individual.

As used herein, the phrase “reducing blood glucose levels” refers to the reduction of abnormally high e.g., above 110 mg/dl in an adult human, levels of blood glucose. Blood glucose levels can be monitored using systems such as the OneTouch® made by LifeScan, Inc. in order to determine suitability for treatment.

Administration of the stem cells of the present invention can be effected using any suitable route such as intravenous, intra peritoneal, intra hepatic, intra spleenic, intra pancreatic, subcutaneous, transcutaneous, intramuscular, intracutaneous, intrathecal, epidural and rectal. According to presently preferred embodiments, the stem cells of the present invention are introduced to the individual using intra hepatic, intra spleenic and/or intra peritoneal administrations.

As is further shown in Example 2 of the Examples section which follows, a single-dose injection of the Pdx1-expressing BM cells into STZ-diabetic immunodeficient (NOD-scid) mice resulted in a long-lasting effect on blood glucose levels, clearly demonstrating that the Pdx1-expressing stem cells of the present invention can be effectively utilized for the treatment of disorders requiring β-cell replacement.

As used herein “treating an individual having a disorder requiring β-cell replacement” refers to treating an individual suffering from a disorder such as diabetes, pancreatic cancer, pancreatitis, and the like that require β-cell replacement.

The phrase “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

Insulin producing stem cells of the present invention cart be generated from stem cells derived from the treated individual (autologous source) or from allogeneic sources. Since non-autologous cells are likely to induce an immune reaction when administered to the body, steps are taken to reduce such reaction when non-autologous cells are utilized. These include either suppressing the recipient immune system or encapsulating the non-autologous cells or tissues in immunoisolating, semipermeable membranes prior to transplantation.

Encapsulation techniques are generally classified as microencapsulation, involving small spherical vehicles or macroencapsulation, involving larger flat-sheet and hollow-fiber membranes (Uludag, H. et al. Technology of mammalian cell encapsulation Adv Drug Deliv Rev. 2000; 42: 29-64).

Methods of preparing microcapsules are well known in the arts and include for example those disclosed by Lu M Z, et al., Cell encapsulation with alginate and alpha-phenoxycinnamylidene acetylated poly(allylamine). Biotechnol Bioeng. 2000, 70: 479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

Microencapsulated insulin producing stem cells of the present invention can be prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Technol. Ther. 2003, 5: 665-8) or its derivatives can also be utilized to encapsulate the insulin producing cells of the present invention. Alginate based microcapsules can be prepared by polyelectrolyte complexation between the polyanions sodium alginate and sodium cellulose sulphate and the polycation poly(methylene-co-guanidine) hydrochloride in the presence of calcium chloride.

Several prior art studies have shown that cell encapsulation is more effective when smaller capsules are used For example, Canaple et al. have shown that quality control, mechanical stability, diffusion properties, and in vitro activities of encapsulated cells improve when capsule size is reduced from 1 mm to 400 μm (Improving cell encapsulation through size control. J Biomater Sci Polym Ed 2002;13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai T. A. Microfabrication technology for pancreatic cell encapsulation. Expert Opin Biol Ther. 2002, 2: 633-46).

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Preparation of β-Like Cells Using Pdx1-Expressing Bone Marrow Stem Cells

In order to test the suitability of mesenchymal stem cells for cell replacement therapy in insulin deficient individuals, bone marrow derived cells were sequentially transfected with the hTERT—and the Pdx1-containing plasmids (SEQ ID NOs:1 and 2, respectively) to generate insulin-producing, β-like cells.

Materials and Experimental Methods

Expansion of mesenchymal stem cells from bone marrow cells—Human adult BM cells were obtained at Laniado Hospital in Israel using approved protocols. Bone marrow cells were fractionated and cultured at low density to favor the expansion of mesenchymal stem cells, essentially as described at Colter D C. et al., (Rapid expansion of recycling stem cells in cultures of plastic-adherent cells from human bone marrow. Proc Natl Acad Sci USA 97: 3213-3218, 2000). Under these conditions, the stem cell population doubling time was approximately two days.

Preparation of hTERT/GFP epression vector—A 3.4 Kb of the catalytic subunit of human telomerase (hTERT) cDNA (SEQ ID NO:1) was ligated into the pcDNA3.1 expression vector (Invitrogen, Carlsbad, Calif.) and was placed under the control of the cytomegalovirus (CMV) promoter, upstream of an encephalomyocarditis virus internal ribosomal entry site (IRES) and an enhanced green fluorescence protein (GFP) gene.

Preparation of Pdx1 expression vector—A 0.8 K-b fragment of the rat Pdx1 cDNA (SEQ ID NO:2) was ligated into the pcDNA3.1 expression vector (Invitrogen, Carlsbad, Calif.) and was placed under control of the mouse phosphoglycerate kinase 1 promoter (PGKₚ), upstream of an IRES element, a neomycin resistance gene, and the post-transcriptional regulatory element of woodchuck hepatitis virus (WHV).

Transfection of bone marrow mesenchymal stem cells with the hTERT/GFP-containing plasmid—Following 10 days in culture the stem cells were transfected with a plasmid containing the hTERT/GFP expression vector (FIG. 2a) using FuGENE 6 Transfection Reagent (Roche, Indianapolis, Ind.).

Selection of GFP-positive bone marrow stem cells using fluorescence-activated cell sorting (FACS)—Five days following transfection with the hTERT/GFP plasmid, 2×10⁶ cells were trypsinized and resuspended in 4 ml Hank's balanced salt solution (Biological Industries, Beth Haemek, Israel). Resuspended cells were sorted for the presence of GFP protein using the FACSort machine (Becton Dickinson, San Jose, Calif.) at a rate of 200-300 cells per second. GFP-positive cells were resuspended in a complete growth medium (AMEM medium supplemented with 20% fetal calf serum, 100 units/ml penicillin, 100 μg/ml streptomycin, and 2 mM L-glutamine; all reagents were from Biological Industries, Beth Haemek, Israel) for further propagation

Sequential transfection of hTERT/GFP-positive bone marrow stem cells with the Pdx1-containing plasmid—hTERT/GFP-transfected—or untransfected—bone marrow stem cells (3×10⁶) were transfected with 5 μl of the Pdx1-containing plasmid DNA (FIG. 2b) mixed with 15 μl of the FMGENE 6 reagent (Roche, Indianapolis, Ind.) according to manufacturer's instructions.

HTERT immunofluorescence—Cells were fixed in 4% paraformaldehyde and incubated overnight at 4° C. with 1:200 dilutions of rabbit-anti-human hTERT serum (Santa Cruz, Calif.) in PBS containing 1% bovine serum albumin (BSA), 10% fetal bovine serum, and 0.2% saponin. Cells were then washed in 1% BSA in PBS, incubated for 1 hour at room temperature with 1:20 dilutions of goat anti-rabbit serum conjugated to rhodamine (Santa Cruz, Calif.) and stained for 10 minutes at room temperature with a solution of 100 ng/ml of DAPI (Roche, Indianapolis, Ind.). Cells were photographed using a fluorescent Nikon microscope (Nikon, Tokyo, Japan).

Insulin immunofluorescence and RT-PCR analyses were performed essentially as described at Zalzman M. et al., (Reversal of hyperglycemia in mice by using human expandable insulin-producing cells differentiated from fetal liver progenitor cells. Proc. Natl. Acad. Sci. USA100: 7253-7258, 2003). PCR primers and PCR-specific annealing temperatures are described in Table 1, hereinbelow.

TABLE 1

PCR primers and conditions

| | | | | |
|---|---|---|---|---|
| Gene product | | | | |
| (Accession | | Forward (F) and reverse | Annealing | Size |
| number) | SEQ ID NOs. | (R) primers (5′→3′) | Temp. | (bp) |

| | | | | |
|---|---|---|---|---|
| rPdx1 | SEQ ID NO:3 | F: CAA GGA CCC GTG CGC ATT CCA GAG G | 60° C. | 454 |
| (NM_022852) | SEQ ID NO:4 | R: GAA CTC CTT CTC CAG CTC TAG CAG CTG | | |
| hPDX1 | SEQ ID NO:5 | F: TCCACCTTGGGACCTGTTTAGAG | 54.9° C. | 232 |
| (NM_000209) | SEQ ID NO:6 | R: GGACTCACTGTATTCCACTGGCATC | | |
| BETA2/ | SEQ ID NO:7 | F: CCT GAG CAG AAC CAG GAC ATG CC | 58° C. | 215 |
| NEUROD1 | SEQ ID NO:8 | R: CTT GAC CTG ACT CTC TGT CAT C | | |
| (NM_002500) | | | | |
| NKX6.1 | SEQ ID NO:9 | F: CTC CTC CTC GTC CTC GTC GTC GTC | 60° C. | 329 |
| (NM_006168) | SEQ ID NO:10 | R: CTT GAC CTG ACT CTC TGT CAT C | | |
| Nkx2.2 | SEQ ID NO:11 | F: CGG ACA ATG ACA AGG AGA CCC CG | 65° C. | 485 |
| (AH007038) | SEQ ID NO:12 | R: CGC TCA CCA AGT CCA CTG CTG CTG G | | |
| ISL1 | SEQ ID NO:13 | F: GTG CGG AGT GTA ATC AGT ATT TGG | 58° C. | 496 |
| (NM_002202) | SEQ ID NO:14 | R: GTC ATC TCT ACC AGT TGC TCC TTC | | |
| Insulin | SEQ ID NO:15 | F: GCT GCA TCA GAA GAG GCC ATC AGG C | 58° C. | 381 |
| (NM_000207) | SEQ ID NO:16 | R: GCG TCT AGT TGC AGT AGT TCT CCA G | | |
| PC1/3 | SEQ ID NO:17 | F: TTG GCT GAA AGA GAA CGG GAT ACA TCT | 65° C. | 457 |
| (NM_000439) | SEQ ID NO:18 | R: ACT TCT TTG GTG ATT GCT TTG GCG GTG | | |
| PC2 | SEQ ID NO:19 | F: GCA TCA AGC ACA GAC CTA CAC TCG | 60° C. | 309 |
| (NM_002594) | SEQ ID NO:20 | R: GAG ACA CAA CCA CCC TTC ATC CTT C | | |
| GLUT1 | SEQ ID NO:21 | F: CTCACTGCTCAAGAAGACATGG | 60° C. | 349 |
| (NM_006516) | SEQ ID NO:22 | R: CTGGGTAACAGGGATCAAACAG | | |
| GLUT2 | SEQ ID NO:23 | F: GCCATCCTTCAGTCTCTGCTACTC | 65° C. | 525 |
| (NM_000340) | SEQ ID NO:24 | R: GCTATCATGCTCACATAACTCATCCA | | |
| GK | SEQ ID NO:25 | F: GAC GAG TTC CTG CTG GAG TAT GAC | 65° C. | 523 |
| (NM_033507) | SEQ ID NO:26 | R: GAC TCG ATG AAG GTG ATC TCG CAG CTG | | |
| SUR1/ | SEQ ID NO:27 | F: GTG CAC ATC CAC CAC AGC ACA TGG CTTC | 60° C. | 429 |
| ATP binding | SEQ ID NO:28 | R: GTG TCT TGA AGA AGATGT ATC TCC TCA C | | |
| (NM_000352) | | | | |
| KIR6.2/ | SEQ ID NO:29 | F: CGCTGGTGGACCTCAAGTGGC | 62.5° C. | 497 |
| KCNJ11 | SEQ ID NO:30 | R: CCTCGGGGCTGGTGGTCTTGCG | | |
| (NM_000525) | | | | |
| IAPP | SEQ ID NO:31 | F: GAG AGA GCC ACT GAA TTA CTT GCC | 65° C. | 471 |
| (NM_000415) | SEQ ID NO:32 | R: CCT GAC CTT ATC GTG ATC TGC CTG C | | |
| SYNG3 | SEQ ID NO:33 | F: GGTAGTGACTGTCTCGTTTCTGTC | 60° C. | 459 |
| (NM_004209) | SEQ ID NO:34 | R: AGCTATGCAGAGGGACTCCAACCTG | | |
| Ngn3 | SEQ ID NO:35 | F: CACCAAACAGCGAAGAAGCC | 59.9° C. | 304 |
| (NM_020999) | SEQ ID NO:36 | R: TTGGGAGACTGGGGAGTAGATAGAG | | |
| PAX4 | SEQ ID NO:37 | F: CAC CTCTCTGCCTGAGGACACGGTGAG | 60° C. | 443 |
| (NM_006193) | SEQ ID NO:38 | R: CTGCCT CATTCCAAGCCATACAGTAGTG | | |
| PAX6 | SEQ ID NO:39 | F: CAGTCACAGCGGAGTGAATCAGC | 58° C. | 562 |
| (NM_001604) | SEQ ID NO:40 | R: GCCATCTTGCGTAGGTTGCCCTG | | |
| Glucagon | SEQ ID NO:41 | F: GAA TTC ATT GCT TGG CTG GTG AAA GGC | 60° C. | 255 |
| (NM_002054) | SEQ ID NO:42 | R: CAT TTC AAA CAT CCC ACG TGG CAT GCA | | |
| PP | SEQ ID NO:43 | F: CTG CTG CTC CTG TCC ACC TGC GTG | 60° C. | 446 |
| (NM_002722) | SEQ ID NO:44 | R: CTC CGA GAA GGC CAG CGT GTC CTC | | |
| Somatostatin | SEQ ID NO:45 | F: CGT CAG TTT CTG CAG AAG TCC CTG GCT | 60° C. | 196 |
| (NM_001048) | SEQ ID NO:46 | R: CCA TAG CCG GGT TTG AGT TAG CAG ATC | | |
| Elastase I | SEQ ID NO:47 | F: GTG ATG ACA GCT GCT CAC TGC GTG | 60° C. | 417 |
| (NM_007352) | SEQ ID NO:48 | R: CAT CTC CAC CAG CAC ACA CCA TGG TG | | |
| HNF3β/ | SEQ ID NO:49 | F: CGC CTT CAA CCA CCC GTTC | 59.9° C. | 361 |
| FOXOA2 | SEQ ID NO:50 | R: CAA CAC CGT CTC CCC AAA GTC | | |
| (NM_021784) | | | | |

Experimental Results

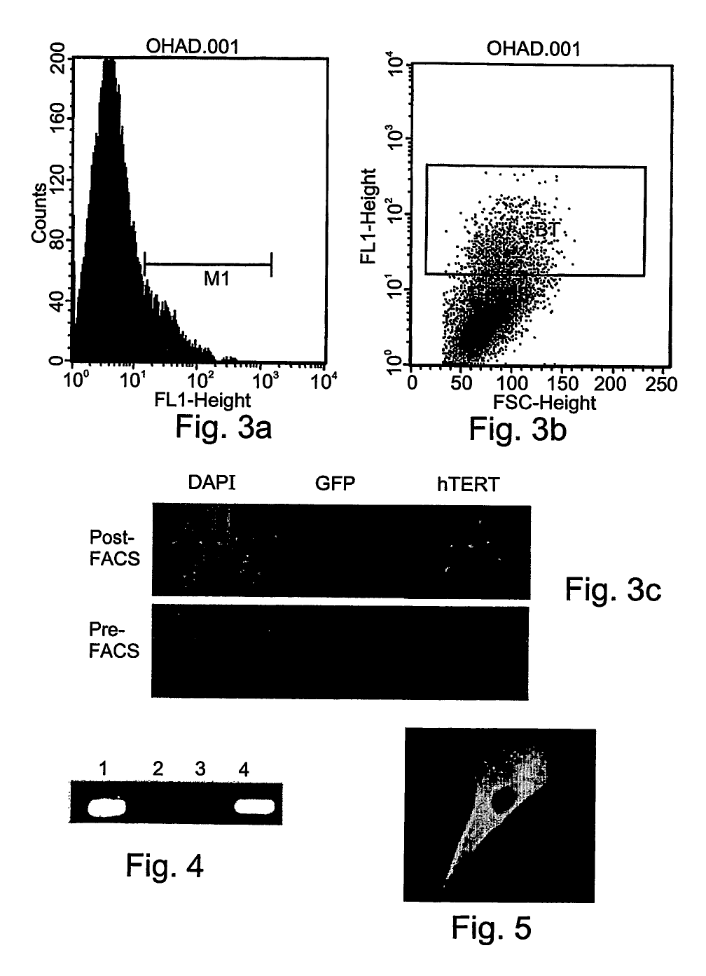

Preparation of hTERT/GFP—positive mesenchymal stem cells—Bone marrow stem cells were cultured under conditions favoring the expansion of mesenchymal stem cells. To increase their culturing capacity and avoid telomere shortening the cells were transfected with the cDNA encoding the catalytic subunit of human telomerase (hTERT) placed under the control of the CMV promoter in a plasmid construct containing the enhanced green fluorescent protein (GFP) as a reporter gene. The level of transfection was measured by FACS analysis using the green fluorescence generated by the expressed GFP gene. As is shown in FIG. 3a, about 20% of the mesenchymal stem cells were successfully transfected with the hTERT/GFP plasmid and expressed the GFP gene.

To enrich the fraction of hTERT/GFP-positive cells, the cells were selected using GFP-guided FACS. As is shown in FIG. 3b, this procedure resulted in a significant enrichment of the hTERT/GFP-positive cells. Noteworthy, hTERT/GFP-positive cells exhibited a doubling time of 2 days, similarly to untransfected bone marrow stem cells.

HTERT/GFP-transfected cells express the hTERT gene—The expression of the hTERT and GFP genes was demonstrated using immunofluorescence Following FACS-selection of GFP-positive cells, the cells were subjected to immunofluorescence analysis using an antibody directed against the hTERT protein. As is shown in FIG. 3c, while prior to FACS-selection most DAPI-stained nucleated cells exhibited negative immunostaining for either GFP or hTERT (FIG. 3c, Pre-FACS), a significant fraction of the nucleated cells were positive for both GFP and hTERT immunostaining post FACS (FIG. 3c).

The expression of hTERT was further demonstrated using RT-PCR analysis. As is shown in FIG. 4, hTERT expression was detected in hTERT/GFP-transfected bone marrow stem cells (FIG. 4, lane 4) but not in untransfected bone narrow stem cells (FIG. 4, lane 3).

Sequential transfection of hTERT/GFP-positive cells with the Pdx1-containing plasmid—To reprogram the mesenchymal stem cells to produce insulin hTERT/GFP-positive cells were further transfected with a plasmid vector containing the rat pancreatic duodenal homeobox 1 (Pdx1) gene (SEQ ID NO:2, GenBank Accession number NM_{—}022852) under the control of the phosphoglycerate kinase (PGK) promoter (FIG. 2b).

Pdx1-expressing cells are capable of producing insulin in culture—To test their ability to produce insulin in culture, Pdx1-expressing bone marrow stem cells were subjected to immunofluorescence analysis using antibodies directed against insulin. As is shown in FIG. 5, six weeks following Pdx1 transfection, Pdx1-expressing cells produced high amounts of insulin. These results demonstrate that bone marrow stem cells are capable of being reprogrammed to insulin-producing cells under the control of the dominant master gene, Pdx1.

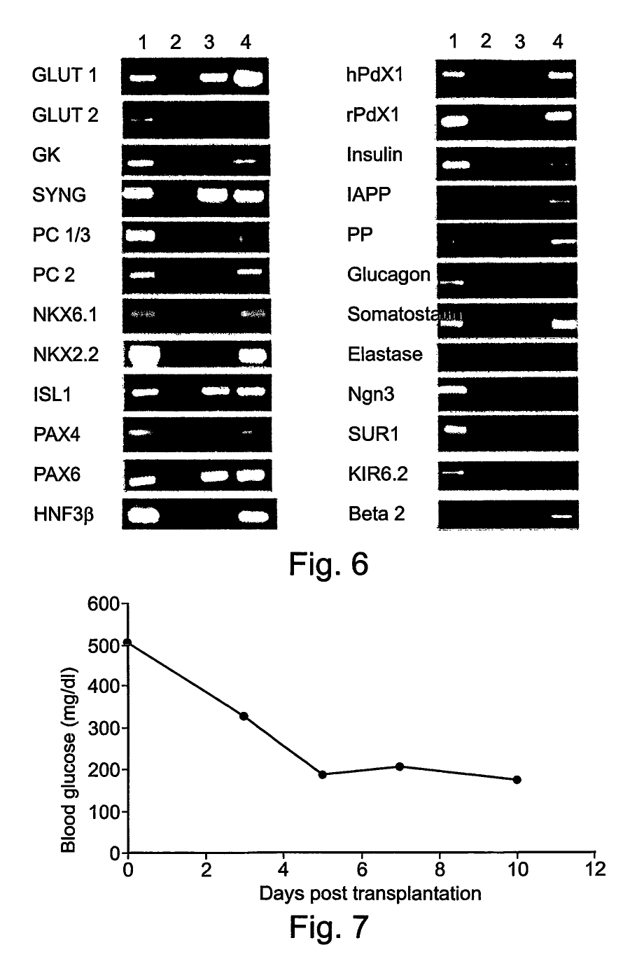

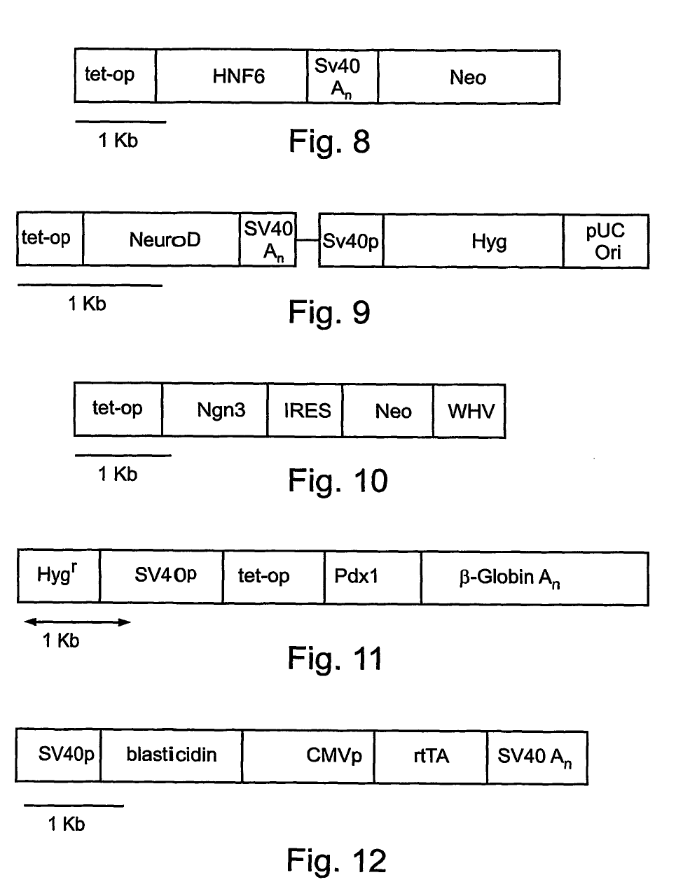

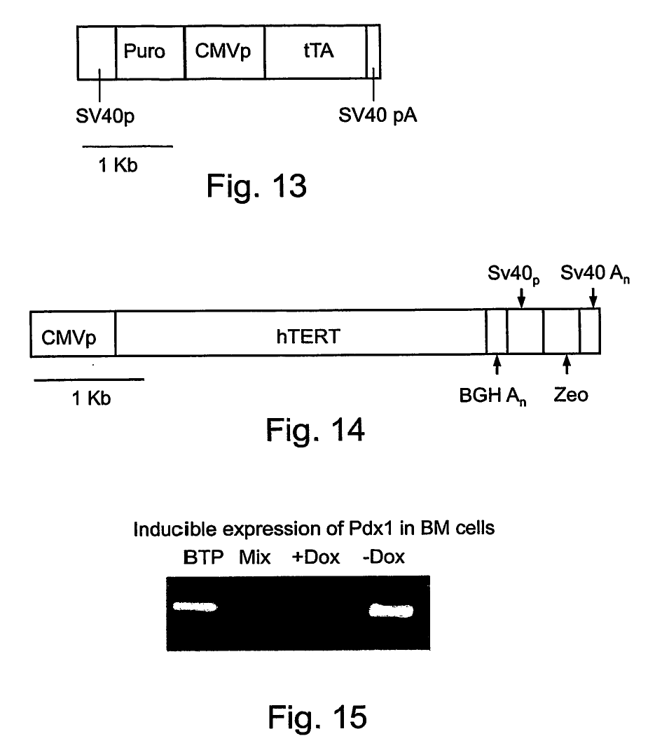

Pdx1-positive cells display characteristic β-like gene expression pattern—RT-PCR analysis was employed in order to evaluate the effect of expressing the rat Pdx1 gene in human bone marrow mesenchymal stem cells. As is shown in FIG. 6, the expression of the rat Pdx1 gene activated the endogenous human Pdx1 gene, as well as the expression of other typical β-cell genes, including insulin, islet amyloid polypeptide (IAPP), glucokinase (GK), glucose transporter member 2 (GLUT2), prohormone convertase type 1 (PC⅓), prohormone convertase type 2 (PC2), homeodomain proteins NKX2.2 and NKX6.1, the K⁺ inward rectifier 6.2 (KIR6.2), and paired box gene 4 (PAX4). In addition, transcripts of genes expressed in other pancreatic cell types were also detected, including glucagon, pancreatic polypeptide (PP), somatostatin and elastase. Moreover, the expression of the transcription factors Beta 2 and HNF3_{β} genes, which are required for normal β-cell gene expression, in Pdx1-expressing bone marrow stem cells suggests their conversion to β-like cells. On the other hand, transcripts of sulfonylurea receptor 1 (SUR1) and neurogenin 3 (Ngn3) genes were not detected. These results demonstrate that under the control of the Pdx1 gene bone marrow mesenchymal stem cell are capable of generating β-like cells.

Altogether, these results demonstrate that Pdx1-expressing bone marrow stem cells are capable of producing insulin and expressing other typical β-cell genes in culture.

### Example 2

### PDX1-Expressing Bone Marrow Stem Cells are Capable of Reducing Blood Glucose Levels In Vivo

In order to assess the capability of the Pdx1-expressing bone marrow stem cells of the present invention to reduce blood glucose levels, these cells were transplanted in a STZ-diabetic immunodeficient (NOD-scid) mouse and the glucose blood levels were determined.

Materials and Experimental Methods

Induction of hyperglycemia in NOD-scid mice—Hyperglycemia was induced in four-month-old nonobese diabetic severe combined immunodeficiency (NOD-scid) male mice (Harlan, Jerusalem, Israel) by intra peritoneal (I.P.) injections of 170 μg per gr body weight of streptozotocin (STZ). Blood glucose levels were measured in samples obtained from the tail vein using the Accutrend strips (F. Hoffman-La Roche Ltd, Basel, Switzerland). Mice were considered hyperglycemic when blood glucose levels reached 300 mg/dl.

Transplantation of Pdx1-expressing bone marrow stem cells into STZ-diabetic NOD-scid mice—Pdx1-expressing bone marrow stem cells (2×10⁶ cells in n 0.2 ml PBS) were injected into the tail vein of an hyperglycemic STZ-MOD-scid mouse.

Experimental Results

Transplantation of bone marrow stem cells in STZ-diabetic NOD-scid mouse—Pdx1-expressing bone marrow stem cells were transplanted via i.v. injection into a STZ-diabetic immunodeficient (NOD-scid) mouse, and the blood glucose levels were determined. As is shown in FIG. 7, on the day of injection the test animal exhibited high levels of blood glucose (500 mg/dl) which are typical of diabetic mice. Five days following a single injection of 2×10⁶ Pdx1-expressing cells the measured blood glucose levels of the tested diabetic mouse significantly dropped to 180 mg/dl, which is within the normal range of fed blood glucose levels in mice. As is further shown in FIG. 7, the measured blood glucose levels remain-ed normal for at least 6 days following normalization by a single injected dose of Pdx1-bone marrow stem cells.

Thus, these results demonstrate that Pdx1-expressing cells of the present invention are capable of reducing blood glucose levels in vivo. Moreover, these results suggest a long-lasting effect of a single-dose injection of Pdx1-expressing bone marrow stem cells. Thus, these results clearly illustrate that the Pdx1-expressing bone marrow stem cells of the present invention are highly suitable for the treatment of diabetes and in particular type 1 diabetes.

### Example 3

### Inducible Expression of PDX1 in Bone Marrow Cells

To enable regulated and time-restricted expression of transcription factors inducing differentiation of stem cells into β-like cells (i.e., insulin-producing cells) various inducible DNA vectors have been constructed, as follows.

Materials and Experimental Methods

Preparation of the tet-HNF6/Neo DNA construct—A 1.6 Kb fragment of the rat hepatocyte nuclear factor 6 (HNF6) cDNA (SEQ ID NO:51) was ligated between the EcoRb1 and HindIII restriction enzyme sites of the pUHD10-3 vector [a gift of H. Bujard, see Efrat S, 1995 (Supra)], which placed it under control of the a minimal promoter (CMV and the tet-operator sequences (tet-op) and upstream of an SV40 polyadenylation element (SV40 Aₙ). This vector also includes a neomycin resistance gene (Neo).

Preparation of the tet-NeuroD/Hyg DNA construct—A 1 kb fragment of the human neurogenic differentiation 1 (NeuroD) cDNA (SEQ ID NO:52) was ligated between the BamHLI and XbaI restriction enzyme sites of the PTRE2Hyg (Clontech) expression vector, which placed it under control of a minimal promoter (CMV promoter) and the tet-operator sequences (tet-op) and upstream of an SV40 polyadenylation element (Aₙ). This vector also contains a hygroycin resistance gene (Hyg).

Preparation of the tet-Ngn3/Neo DNA construct—A 750 bp fragment of the mouse neurogenin 3 (Ngn3) cDNA (SEQ ID NO:53) was ligated between the HindIII and XbaI restriction enzyme sites of the pUHD10-3 vector [Efrat S, 1995 (Supra)], which placed it under control of a minimal promoter (CMV promoter) and the tet-operator sequences (tet-op). The tet-Ngn3 fragment was then removed (using the AatII and XbaI restriction enzymes) and ligated upstream of an IRES (internal ribosome entry site) element of the pIRES (Clontech) vector, which also includes a neomycin resistance gene (Neo) and a woodchuck hepatitis virus posttranscriptional modification element (WHV).

Preparation of the tet-Pdx1/Hyg DNA construct—A 800 bp fragment of the Rat Pdx1 cDNA (SEQ ID NO:2) was ligated between the BamHI and ClaI restriction enzyme sites of the PTRE2Hyg expression vector (Clontech), which placed it under control of a minimal promoter and the tet-operator sequences (tet-op) and upstream of a β-globin polyadenylation element (P3-globin Aₙ).

Preparation of the CMV-rtTA/Bla DNA construct—was purchased from Invitrogene (vector name: PcDNA6/TR).

Preparation of the CMV-tTA/Puro DNA construct—The 1.1 Kb fragment including a Purocyclin resistance gene under control of a SV40 promoter was ligated into the XhoI restriction enzyme site of the pUHD-15-1 vector.

Preparation of the CMV-hTERT/Zeo construct—The human telomerase reverse transcriptase (hTERT) cDNA (SEQ ID NO:1) was ligated between the BamHI and XhaI restriction enzyme sites of the PcDNA3.1 Zeo expression vector (Invitrogen), which placed it under control of the CMV promoter and upstream of a bovine growth hormone (BGH) polyadenylation site.

Experimental Results

Regulated Expression of the Pdx1 Gene Using the tet-Off Transactivator System—In order to prepare regulated β-like insulin-producing cells, bone marrow cells were co-transfected with the tet-off transactivator system (i.e., the CMV-tTA/Puro and tet-Pdx1/Hyg expression vectors) which activates transcription of Pdx1 only in the absence of tetracycline. Transfected cells were cultured for three days in the presence or absence of 0.5 μg/ml Doxycycline. As is shown in FIG. 15, cells co-transfected with the tet-off transactivator system expressed Pdx1 only in the absence of tetracycline (Doxycycline). These results suggest the use of the tet-off transactivator system to regulate the expression of Pdx1 and thus to control the production of insulin in such cells.

Regulated expression of dell transcription factors is expected to generate more efficient insulin production from transfected stem cells—Using the tet-off transactivator system (CMV-TA/Puro) and the tet-Pdx1/Hyg and/or the tet-NeuroD/Hyg DNA vectors, transfected cells are treated with tetracycline for a limited time period (e.g., for 10 days) which enables efficient cell expansion in the absence of differentiation factors (e.g., Pdx1 and NeuroD). Once the cell culture is efficiently expanded, tetracycline is removed from the culture and the transcription factors (Pdx1 and/or NeuroD) are expressed and promote an efficient β-like insulin-production in the transfected stem cells.

Limited expression of BNF6 and Ngn3 is expected to promote differentiation of stem cells into β-like precursor cells—Since the HNF6 and Ngn3 differentiation factors are known to promote differentiation of stem cells into endocrine progenitors, but not to β-like cells (Gu G, et al., 2002. Development, 129: 2447-57), a transient expression of such differentiation factors is desired in order to promote differentiation of stem cells into endocrine progenitors. Such transient expression can be accomplished using the tet-on reverse transactivator system (CMV-rtTA/Bla) and the tet-HNF6/Neo and/or the tet-Ngn3/Neo DNA vectors. Transfected cells are treated with doxycycline for a limited time period (e.g., 2-3 weeks) following which doxycycline is removed from the culture and the endocrine progenitors can further differentiate into β-like cells in the absence of HNF6 and/or Ngn3.

The CMV-hTERT/Zeo DNA construct with a selectable marker (i.e., Zeomycin resistance gene) was constructed to replace the hTERT/GFP construct (which is described in Example 1, hereinabove) to allow easier selection of transfected cells.

Altogether, the new vectors described herein can be used to achieve regulated and reversible expression of various transcription factors which promote differentiation of the stem cells of the present invention into β-like insulin-producing cells.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and GenBank Accession numbers mentioned in this specification are herein incorporated in their entirety by reference into the specification, to the same extent as if each individual publication, patent, patent application or GenBank Accession number was specifically and individually indicated to be incorporated herein by reference. In addition, citation or identification of any reference in this application shall not be construed as an admission that such reference is available as prior art to the present invention.

1-74. (canceled)

75. A method of generating insulin-producing cells comprising:
(a) isolating and optionally culturing adult tissue stem cells to thereby obtain an adult tissue stem cell culture; and
(b) expressing exogenous Pdx1 in cells of said adult tissue stem cell culture to thereby obtain insulin-producing cells.

76. The method of claim 75, further comprising a step of immortalizing said cells of said adult tissue stem cell culture.

77. The method of claim 76, wherein said immortalizing is effected by expressing in said cells of said adult tissue stem cell culture a telomerase.

78. The method of claim 75, wherein said adult tissue stem cells are selected from the group consisting of adipose tissue stem cells, skin stem cells, kidney stem cells, liver stem cells, prostate stem cells, pancreas stem cells, intestine stem cells and bone marrow stem cells.

79. The method of claim 78, wherein said adult tissue stem cells are bone marrow stem cells.

80. The method of claim 75, wherein said culturing is effected under culturing conditions selected suitable for expansion of mesenchymal stem cells of said bone marrow stem cells.

81. The method of claim 75, wherein said expressing in said cells said exogenous Pdx1 is effected by transfecting said cells with an expression vector including a polynucleotide encoding said Pdx1 positioned under the transcriptional control of a mammalian promoter.

82. A cell culture comprising adult tissue stem cells genetically modified to express Pdx 1.

83. The cell culture of claim 82, wherein said adult tissue stem cells are selected from the group consisting of adipose tissue stem cells, skin stem cells, kidney stem cells, liver stem cells, prostate stem cells, pancreas stem cells, intestine stem cells and bone marrow stem cells.

84. The cell culture of claim 82, wherein said adult tissue stem cells are bone marrow stem cells.

85. The cell culture of claim 82, wherein said cells are capable of producing insulin.

86. The cell culture of claim 82, wherein said cells are capable of expressing pancreatic beta cell genes.

87. The cell culture of claim 82, wherein said adult tissue stem cells stem cells are immortalized.

88. The cell culture of claim 87, wherein said adult tissue stem cells stem cells are genetically modified to express a telomerase.

89. A method of reducing blood glucose levels in an individual comprising administering Pdx1-expressing adult tissue stem cells to the individual thereby reducing blood glucose levels in the individual.

90. The method of claim 89, wherein said Pdx1-expressing bone marrow stem cells are prepared by:
(a) isolating and optionally culturing adult tissue stem cells to thereby obtain an adult tissue stem cell culture; and
(b) expressing exogenous Pdx1 in cells of said adult tissue stem cell culture to thereby obtain Pdx1-expressing bone marrow stem cells.

91. The method of claim 90, further comprising a step of immortalizing said cells of said adult tissue stem cell culture.

92. The method of claim 91, wherein said immortalizing is effected by expressing in said cells of said adult tissue stem cell culture a telomerase.

93. The method of claim 89, wherein said adult tissue stem cells are selected from the group consisting of adipose tissue stem cells, skin stem cells, kidney stem cells, liver stem cells, prostate stem cells, pancreas stem cells, intestine stem cells and bone marrow stem cells.

94. The method of claim 89, wherein said adult tissue stem cells are bone marrow stem cells.

95. The method of claim 94, wherein said culturing is effected under culturing conditions selected suitable for expansion of mesenchymal stem cells of said bone marrow stem cells.

96. The method of claim 90, wherein said expressing in said cells said exogenous Pdx1 is effected by transfecting said cells with an expression vector including a polynucleotide encoding said Pdx1 positioned under the transcriptional control of a mammalian promoter.

97. A method of treating an individual having a disorder requiring β-cell replacement, comprising administering Pdx1-expressing adult tissue stem cells to the individual thereby treating the individual.

98. The method of claim 97, wherein said Pdx1-expressing adult tissue stem cells are prepared by:
(a) isolating and optionally culturing adult tissue stem cells to thereby obtain an adult tissue stem cell culture; and
(b) expressing exogenous Pdx1 in cells of said adult tissue stem cell culture to thereby obtain Pdx 1-expressing adult tissue stem cells.

99. The method of claim 98, further comprising a step of immortalizing said cells of said adult tissue stem cell culture.

100. The method of claim 99, wherein said immortalizing is effected by expressing in said cells of said adult tissue stem cell culture a telomerase.

101. The method of claim 97, wherein said adult tissue stem cells are selected from the group consisting of adipose tissue stem cells, skin stem cells, kidney stem cells, liver stem cells, prostate stem cells, pancreas stem cells, intestine stem cells and bone marrow stem cells.

102. The method of claim 97, wherein said adult tissue stem cells are bone marrow stem cells.

103. The method of claim 102, wherein said culturing is effected under culturing conditions selected suitable for expansion of mesenchymal stem cells of said bone marrow stem cells.

104. The method of claim 98, wherein said expressing in said cells said exogenous Pdx1 is effected by transfecting said cells with an expression vector including a polynucleotide encoding said Pdx1 positioned under the transcriptional control of a mammalian promoter.
